(19) United States
(12) Patent Application Publication
(10) Pub. No.: US 2012/0196850 A1
(43) Pub. Date: Aug. 2, 2012

(54) ASSOCIATION OF A SINUS NODE If CURRENT INHIBITOR AND AN ANGIOTENSIN CONVERTING ENZYME INHIBITOR, AND PHARMACEUTICAL COMPOSITIONS CONTAINING IT

(75) Inventors: Vidal BENATAR, Saint-Cloud (FR); Guy LEREBOURS-PIGEONNIERE, Levallois Perret (FR)

(73) Assignee: LES LABORATOIRES SERVIER, Suresnes Cedex (FR)

(21) Appl. No.: 13/442,213

(22) Filed: Apr. 9, 2012

Related U.S. Application Data
(63) Continuation of application No. 11/642,899, filed on Dec. 20, 2006.

(30) Foreign Application Priority Data
Dec. 21, 2005 (FR) 05.13006

(51) Int. Cl. A61K 31/55; A61P 9/12

(52) U.S. Cl. 514/212.07

(57) ABSTRACT

Association comprising a selective and specific sinus node I_{f} current inhibitor, more especially ivabradine, and an agent that inhibits angiotension-converting enzyme.

Medicinal products containing the same which are useful in treating arterial hypertension.

The present invention relates to a new association of a selective and specific sinus node I_{f} current inhibitor and an agent that inhibits angiotensin-converting enzyme. More specifically, the present invention relates to a new association of a selective and specific sinus node I_{f} current inhibitor which is ivabradine, or 3-{3-[{[(7S)-3,4-dimethoxy-bicyclo[4.2.0]octa-1,3,5-trien-7-yl]methyl}(methyl)amino]propyl}-7,8-dimethoxy-1,3,4,5-tetrahydro-2H-3-benzazepin-2-one, of formula (I):

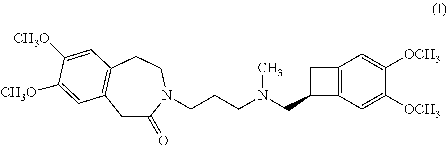

and its hydrates and crystalline forms and addition salts thereof with a pharmaceutically acceptable acid, and an agent that inhibits angiotensin-converting enzyme.

Selective and specific sinus node I_{f} current inhibitors, more especially ivabradine, and its hydrates and crystalline forms and addition salts thereof with a pharmaceutically acceptable acid, more especially its hydrochloride, have very valuable pharmacological and therapeutic properties, especially negative chronotropic properties (lowering of heart rate), which make these compounds useful in the treatment, prevention and prognosis improvement of various cardiovascular diseases associated with myocardial ischaemia such as angina pectoris, myocardial infarct and associated rhythm disturbances, and also in various pathologies involving rhythm disturbances, especially supraventricular rhythm disturbances, and in chronic heart failure.

The preparation and therapeutic use of ivabradine and addition salts thereof with a pharmaceutically acceptable acid, more especially its hydrochloride, have been described in the European patent specification EP 0 534 859.

The Applicant has now discovered, surprisingly, that selective and specific sinus node I_{f} current inhibitors, more especially ivabradine, or 3-{3-[{[(7S)-3,4-dimethoxybicyclo-[4.2.0]octa-1,3,5-trien-7-yl]methyl}(methyl)amino]propyl}-7,8-dimethoxy-1,3,4,5-tetra-hydro-2H-3-benzazepin-2-one, used in association with an agent that inhibits angiotensin-converting enzyme, have valuable properties which allow them to be used in association in the treatment of arterial hypertension.

Arterial hypertension is a silent, but insidious disease: although for most of the time it is not accompanied by any immediate problems, it makes itself manifest, when untreated, after 10 to 20 years, by the occurrence of a serious vascular, cardiac or cerebral accident. Beyond a biological parameter defined as exceeding a particular level which is determined by experts, arterial hypertension is a major risk factor for cardiovascular diseases, these diseases representing the most common cause of death in the last third of life in people in industrial countries.

This health “time bomb” accordingly poses a very great public health problem in view of its high incidence among the general population. From the therapeutic point of view, hygienic-dietary measures are always necessary. These make it possible sometimes to avoid, but most often simply to delay, use of drug treatment. The arsenal of drugs is large but is still frequently inadequate for obtaining satisfactory control of arterial pressure.

It is known that the level of arterial pressure (systolic or diastolic) is a very important determining factor in the risk of occurrence of a cerebral vascular accident or myocardial infarct. A very large number of clinical trials have shown that lowering arterial pressure very significantly reduces the risk of cerebral and cardiac accidents in hypertensive patients.

It is known that the individual response to an antihypertensive treatment is variable and difficult to predict. Obtaining optimum control of arterial pressure requires recourse to multitherapy (using drugs having different modes of action) in the majority of hypertensive patients.

Compounds are still being sought which are active in forms of hypertension that are resistant to current treatments. In addition, drugs are also sought which have a longer duration of action or which have even fewer secondary clinical or biological effects. Accordingly, the development of new, alternative treatments is still relevant today and remains a necessity.

A therapeutic class widely used in the treatment of arterial hypertension comprises angiotensin-converting enzyme inhibitors (ACE inhibitors).

Angiotensin-converting enzyme inhibitors are one of the major therapeutic classes in the treatment of arterial hypertension. They act principally by inhibiting the synthesis of angiotensin II and by blocking the breakdown of bradykinin.

Their haemodynamic effects fundamentally consist of lowering total peripheral resistance by means of arteriolar vasodilation, which brings about a lowering of arterial pressure without sympathetic stimulation and without water/sodium retention. They are effective in all types of arterial hypertension. In addition to the lowering of arterial pressure, they have been shown to improve the morbidity (myocardial infarct, cerebral vascular accidents) and cardiovascular mortality of hypertensive patients, diabetic patients and patients with pre-existing coronary disease.

They are generally very well tolerated apart from the occurrence in some patients of a dry cough which is reversible on cessation of treatment.

The Applicant has now discovered, surprisingly, that selective and specific sinus node I_{f} current inhibitors, more especially ivabradine, are capable of potentiating the effects of agents that inhibit angiotensin-converting enzyme. This effect, which was not to be foreseen because of the very fact of therapeutic class to which selective and specific sinus node I_{f} current inhibitors belong, makes it possible to consider using the association according to the invention in the treatment of arterial hypertension, and more especially this potentiation should make it to possible to use the association according to the invention in the treatment of patients not controlled by customary therapeutic associations.

Without implying any limitation, the ACE inhibitors which can be used in accordance with the invention are: perindopril, captopril, enalapril, lisinopril, delapril, fosinopril, quinapril, ramipril, spirapril, imidapril, trandolapril, benazepril, cilazapril and temocapril, and also their hydrates, crystalline forms and addition salts with a pharmaceutically acceptable acid or base.

Preferred ACE inhibitors are perindopril, captopril, enalapril, ramipril, lisinopril, benazapril, quinapril and delapril, and also their hydrates, crystalline forms and addition salts with a pharmaceutically acceptable acid or base, and more particularly perindopril, or one of its hydrates, crystalline forms and addition salts with a pharmaceutically acceptable acid or base, more especially its tert-butylamine or arginine salt.

The selective and specific sinus node I_{f} current inhibitors are ivabradine and YM758 from Astellas, and also their hydrates, crystalline forms and addition salts with a pharmaceutically acceptable acid or base.

The invention relates more especially to the association of ivabradine, or one of its hydrates, crystalline forms and addition salts with a pharmaceutically acceptable acid, more especially its hydrochloride, and an agent that inhibits angiotensin-converting enzyme, or one of its hydrates, crystalline forms and addition salts with a pharmaceutically acceptable acid.

The invention relates more especially to the association between ivabradine, or one of its hydrates, crystalline forms and addition salts with a pharmaceutically acceptable acid, more especially its hydrochloride, and perindopril, or one of its hydrates, crystalline forms and addition salts with a pharmaceutically acceptable base, more especially its arginine or tert-butylamine salt.

The invention relates also to pharmaceutical compositions comprising the association of a selective and specific sinus node I_{f} current inhibitor and an agent that inhibits angiotensin-converting enzyme, in combination with one or more pharmaceutically acceptable excipients.

The invention relates more especially to pharmaceutical compositions comprising the association of a selective and specific sinus node I_{f} current inhibitor which is ivabradine, or its hydrates, crystalline forms and addition salts with a pharmaceutically acceptable acid, more especially its hydrochloride, and an agent that inhibits angiotensin-converting enzyme, or one of its hydrates, crystalline forms and addition salts with a pharmaceutically acceptable acid, in combination with one or more pharmaceutically acceptable excipients.

The invention relates preferably to pharmaceutical compositions comprising the association of a selective and specific sinus node I_{f} current inhibitor which is ivabradine, or its hydrates, crystalline forms and addition salts with a pharmaceutically acceptable acid, more especially its hydrochloride, and an agent that inhibits angiotensin-converting enzyme which is perindopril, or one of its hydrates, crystalline forms and addition salts with a pharmaceutically acceptable base, more especially its arginine or tert-butylamine salt, in combination with one or more pharmaceutically acceptable excipients.

Amongst the pharmaceutical compositions according to the invention, there may be mentioned, more especially, those that are suitable for oral, parenteral or nasal administration, tablets, dragées, sublingual tablets, capsules, lozenges, suppositories, creams, ointments, dermal gels etc. and also pharmaceutical compositions having programmed, delayed, prolonged or deferred release.

Besides the selective and specific sinus node I_{f} current inhibitor and the compound that inhibits angiotensin-converting enzyme, the pharmaceutical compositions according to the invention comprise one or more excipients or carriers selected from diluents, lubricants, binders, disintegration agents, absorbents, colourants, sweeteners etc.

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the sex, age and weight of the patient, the administration route, the nature of the disorder and of any associated treatments and ranges from 1 to 500 mg of ivabradine per 24 hours and, more preferably, from 10 to 15 mg per day and, also preferably, from 5 to 15 mg per day. The dose of the agent that inhibits angiotensin-converting enzyme may be less than that used when it is administered on its own.

The following Examples illustrate the invention but do not limit it in any way.

### Pharmaceutical Compositions:

Preparation formula for 1000 tablets each containing 7.5 mg of ivabradine and 2 mg of perindopril:

Ivabradine hydrochloride . . . 7.5 g

Perindopril tert-butylamine . . . 2 g

Lactose monohydrate . . . 62 g

Magnesium stearate . . . 1.3 g

Povidone . . . 9 g

Anhydrous colloidal silica . . . 0.3 g

Cellulose sodium glycolate . . . 30 g

Stearic acid . . . 2.6 g

Other examples of pharmaceutical compositions according to the invention are given hereinbelow, without implying any limitation:

### EXAMPLE 1

| Constituents | Amount (mg) |
|---|---|
| ivabradine | 10 |
| perindopril tert-butylamine salt | 4 |

### EXAMPLE 2

| Constituents | Amount (mg) |
|---|---|
| ivabradine | 10 |
| perindopril tert-butylamine salt | 8 |

### EXAMPLE 3

| Constituents | Amount (mg) |
|---|---|
| ivabradine | 15 |
| perindopril tert-butylamine salt | 4 |

### EXAMPLE 4

| Constituents | Amount (mg) |
|---|---|
| ivabradine | 15 |
| perindopril tert-butylamine salt | 8 |

### EXAMPLE 5

| Constituents | Amount (mg) |
|---|---|
| ivabradine | 10 |
| perindopril arginine salt | 5 |

### EXAMPLE 6

| Constituents | Amount (mg) |
|---|---|
| ivabradine | 10 |
| perindopril arginine salt | 10 |

### EXAMPLE 7

| Constituents | Amount (mg) |
|---|---|
| ivabradine | 15 |
| perindopril arginine salt | 5 |

### EXAMPLE 8

| Constituents | Amount (mg) |
|---|---|
| ivabradine | 15 |
| perindopril arginine salt | 10 |

The dosage for the pharmaceutical compositions described above consists of the administration, per os, of one tablet per 24 hours.

In the populations at risk, corresponding to patients more than 75 years old, the initial critical dose administered by the oral route is 5 mg of ivabradine and 2 mg of perindopril tert-butylamine salt or 5 mg of ivabradine and 2.5 mg of perindopril arginine salt per 24 hours in the form of a tablet.

### Clinical Study No. 1:

A clinical study was carried out in 8 patients with arterial hypertension already being treated with an angiotensin-converting enzyme inhibitor (perindopril n=3, ramipril n=2, enalapril n=1, lisinopril n=1, fosinopril n=1) and mild to moderate heart failure of class 1 or 2 according to the NYHA classification. These patients were administered ivabradine at a dose of 7.5 mg twice a day in the case of 7 of them and 5 mg twice a day in the case of 1 of them. After 2 months of treatment (the duration considered sufficient to reach the maximum effect of the two treatments), the mean recumbent systolic and diastolic arterial pressures were lowered by 7.5 mmHg and 7.3 mmHg, respectively. Moreover, taking into consideration those patients who, on inclusion in the study, had an arterial pressure which was not well controlled by the angiotensin-converting enzyme inhibitor, that is to say those who still had a systolic arterial pressure 140 mmHg and/or a diastolic arterial pressure 90 mmHg, the lowering of arterial pressure in that group was 10 mmHg for systolic arterial pressure and 8 mmHg for diastolic arterial pressure.

TABLE 1
Change in lying-down arterial pressure on inclusion and after 2 months
of treatment with an ACE inhibitor and ivabradine

| | Inclusion | 2 months |
|---|---|---|
| SAP (mmHg) | 149.5 | 141.8 |
| DAP (mmHg) | 81.7 | 74.4 |

SAP: Systolic arterial pressure, lying down
DAP: Diastolic arterial pressure, lying down

TABLE 2
Change in lying-down arterial pressure on inclusion and after 2 months
of treatment in patients with arterial pressure
that was not being controlled on entry into the trial.

| | Inclusion | 2 months |
|---|---|---|
| SAP (mmHg) | 159 | 149 |
| DAP (mmHg) | 85 | 77 |

SAP: Systolic arterial pressure, lying down
DAP: Diastolic arterial pressure, lying down

There was accordingly observed a substantial lowering of arterial pressure when ivabradine was added to an angiotensin-converting enzyme inhibitor. This lowering of arterial pressure was unexpected because the mean reduction established in clinical trials with ivabradine is generally of the order of 1 to 2 mmHg for diastolic arterial pressure actually accompanied by a slight increase in systolic arterial pressure of the order of 1 mmHg. This reduction is important because it is known that a reduction of 4 to 5 mmHg in hypertensive patients reduces very substantially (30%) the occurrence of cardiac and neurological accidents.

### Clinical Study No. 2

A complementary clinical study was carried out in patients with arterial hypertension and also severe heart failure of class 3 according to the NYHA classification, corresponding to a cardiopathy which gives rise to marked limitation of physical activity and is comfortable only at rest. The patients were treated over a duration of 6 weeks, the duration recognised as being sufficient to observe clearly the effect of the treatments. The patients received the following treatment, by administration per os:
ivabradine at a dose of 2.5 mg twice a day for 2 weeks; then
for the following 2 weeks, ivabradine at a dose of 5 mg twice a day, except for patients showing a lack of tolerance for the product, such as excessive bradycardia or clinical symptoms associated with marked bradycardia; then
for the last 2 weeks, ivabradine at a dose of 7.5 mg twice a day, except for patients showing a lack of tolerance for the product, such as excessive bradycardia or clinical symptoms associated with marked bradycardia.

40 patients already being treated with an angiotensin-converting enzyme inhibitor such as perindopril, ramipril, enalapril, lisinopril or fosinopril received the treatment described above. The mean recumbent systolic and diastolic arterial pressures were lowered by 2.5 mmHg and 3.8 mmHg, respectively.

TABLE 3
Change in lying-down arterial pressure on inclusion and after 6 weeks
of treatment with an ACE inhibitor and ivabradine

| | Inclusion | 6 weeks |
|---|---|---|
| SAP (mmHg) | 126.3 | 123.8 |
| DAP (mmHg) | 78.4 | 74.6 |

SAP: Systolic arterial pressure, lying down
DAP: Diastolic arterial pressure, lying down

Moreover, 11 patients who, on inclusion in the study, had an arterial pressure which was not well controlled by the angiotensin-converting enzyme inhibitor, that is to say those who still had a systolic arterial pressure ≧140 mmHg and/or a diastolic arterial pressure ≧90 mmHg, received the treatment described above, and the lowering of arterial pressure observed in this group was 10.7 mmHg for systolic arterial pressure and 8.6 mmHg for diastolic arterial pressure.

TABLE 4
Change in lying-down arterial pressure on inclusion and after 6 Weeks
of treatment with an ACE inhibitor and ivabradine, in patients with
arterial pressure that was not being controlled on inclusion

| | Inclusion | 6 weeks |
|---|---|---|
| SAP (mmHg) | 142.1 | 131.4 |
| DAP (mmHg) | 87.4 | 78.8 |

SAP: Systolic arterial pressure, lying down
DAP: Diastolic arterial pressure, lying down

A group of 6 patients already being treated with perindopril as a specific inhibitor of angiotensin-converting enzyme received the treatment described above. A significant lowering of arterial pressure was observed at the end of 6 weeks of treatment: a lowering of systolic arterial pressure by 17.5 mmHg and of diastolic arterial pressure by 7.7 mmHg. In conformity with the conclusions of the preceding clinical study, this is considered a very great reduction in systolic and diastolic arterial pressure in view of the fact that a reduction of 4 to 5 mmHg in hypertensive patients reduces very substantially (30%) the occurrence of cardiac and neurological accidents.

TABLE 5
Change in lying-down arterial pressure on inclusion and after 6 weeks
of treatment with perindopril and ivabradine

| | Inclusion | 6 weeks |
|---|---|---|
| SAP (mmHg) | 127 | 109.5 |
| DAP (mmHg) | 77.5 | 69.8 |

SAP: Systolic arterial pressure, lying down
DAP: Diastolic arterial pressure, lying down

1. A composition comprising a combination of ivabradine, or one of its crystalline forms or addition salts with a pharmaceutically acceptable acid, and an an angiotensin converting enzyme (ACE) inhibitor.

2. The composition of claim 1, wherein the composition comprises a combination of ivabradine hydrochloride, or one of its crystalline forms, and an ACE inhibitor.

3. The composition of claim 1, wherein the ACE inhibitor is perindopril, or one of its crystalline forms or addition salts with a pharmaceutically acceptable base.

4. The composition of claim 1, wherein the ACE inhibitor is perindopril tert-butylamine or arginine salt, or a crystalline form thereof.

5. The composition of claim 1, wherein the composition comprises ivabradine hydrochloride, or one of its crystalline forms, and perindopril tert-butylamine or arginine salt, or a crystalline form thereof.

6. A pharmaceutical composition comprising as active ingredient the composition of claim 1, alone or in combination with one or more pharmaceutically acceptable excipients.

7. The pharmaceutical composition of claim 6, comprising as active ingredient a composition comprising a combination of ivabradine hydrochloride, or one of its crystalline forms, and perindopril tert-butylamine or arginine salt, or a crystalline form thereof.

8. A method for treating a living animal body afflicted with arterial hypertension, comprising the step of administering to the living animal body an amount of the composition of claim 1 which is effective for treatment of arterial hypertension.

9. The method of claim 8, wherein the living animal body is a human.
